(19) United States
(12) Patent Application Publication
(10) Pub. No.: US 2023/0218176 A1
(43) Pub. Date: Jul. 13, 2023

(54) APPARATUS FOR MEASURING BIOMETRIC INFORMATION OF PET

(71) Applicant: BANDAL COMPANY, Seoul (KR)

(72) Inventor: Soyoon SEO, Seoul (KR)

(73) Assignee: BANDAL COMPANY, Seoul (KR)

(21) Appl. No.: 17/928,238

(22) PCT Filed: May 27, 2021
(86) PCT No.: PCT/KR2021/006605

§ 371 Date: Nov. 28, 2022

(30) Foreign Application Priority Data
May 28, 2020 (KR) 10-2020-0064151
Jan. 25, 2021 (KR) 10-2021-0009862

(51) Int. Cl. A61B 5/0205; A61B 5/11; A61B 5/00

(52) U.S. Cl. CPC A61B 5/0205; A61B 5/11; A61B 5/7203

(57) ABSTRACT

Disclosed is a measurement apparatus for measuring biometric information of a pet. The measurement apparatus comprises: a vital sensor configured to measure a vital sign of the pet and generate vital data indicating the vital sign; a motion sensor configured to measure movement of the pet and generate movement data indicating the movement; a memory configured to store the vital data and the movement data; a communication circuit configured to transmit the vital data and the movement data to an external apparatus; and a controller configured to control the measurement apparatus, wherein the controller controls an operation mode of the vital sensor to be any one of an active mode and an inactive mode, on the basis of the movement data measured by the motion sensor, and the vital sensor does not generate the vital data in the inactive mode.

### TECHNICAL FIELD

Embodiments of the present invention relates to an apparatus for measuring biometric information of a pet.

### BACKGROUND ART

Due to the recent increase in a nuclear family and a single person household, the number of pets raised by people is increasing. In addition, interest in a health condition of a pet is increasing, and the scale of related industries is also increasing.

There is an apparatus for measuring biometric information indicating a health condition of a pet, but as the conventional measurement apparatus, a measurement apparatus for humans has adopted for pets. Accordingly, a measurement apparatus suitable for pets is required.

### DISCLOSURE

### Technical Problem

The present disclosure provides an apparatus for measuring biometric information of a pet.

### Technical Solution

According to an embodiment of the present disclosure, a measurement apparatus for measuring biometric information of a pet comprises: a vital sensor configured to measure a vital sign of the pet and generate vital data indicating the vital sign; a motion sensor configured to measure movement of the pet and generate movement data indicating the movement; a memory configured to store the vital data and the movement data; a communication circuit configured to transmit the vital data and the movement data to an external apparatus; and a controller configured to control the measurement apparatus, in which the controller controls an operation mode of the vital sensor to be any one of an active mode and an inactive mode, on the basis of the movement data measured by the motion sensor, and the vital sensor does not generate the vital data in the inactive mode.

### Advantageous Effects

According to embodiments of the present invention, it is possible to accurately measure biometric information related to a pet's living body.

In addition, according to embodiments of the present invention, when movement of a pet is relatively small, a measurement apparatus may measure a vital sign, and when the movement of the pet is relatively large, the measurement apparatus may not measure the vital sign, so it is possible to acquire only vital data that accurately represents a status of a pet. Therefore, it is possible to increase the accuracy of the measurement apparatus.

In addition, according to embodiments of the present invention, when the movement of the pet is relatively large, an operation of a vital sensor may be inactive, so it is possible to reduce the power consumption of the measurement apparatus and further increase the operation time of the measurement apparatus. Therefore, it is possible to maximize the portability of the measurement apparatus.

### DESCRIPTION OF DRAWINGS

FIG. 1 is a diagram illustrating a diagnostic system according to embodiments of the present invention.

FIG. 2 is a diagram illustrating a measurement apparatus according to embodiments of the present invention.

FIG. 3 is a diagram illustrating a part of the measurement apparatus according to embodiments of the present invention.

FIG. 4 is a diagram for describing an operation of a controller according to embodiments of the present invention.

FIG. 5 is a diagram for describing the operation of the controller according to embodiments of the present invention.

FIG. 6 is a flowchart illustrating a method of measuring a heart rate according to embodiments of the present invention.

### MODE FOR CARRYING OUT THE INVENTION

Hereinafter, embodiments of the present invention will be described with reference to the accompanying drawings.

FIG. 1 is a diagram illustrating a diagnostic system according to embodiments of the present invention. Referring to FIG. 1, a diagnostic 10 may include a measurement apparatus 100, a user terminal 200, and a server 300.

The measurement apparatus 100 may be in contact with a pet PET to measure biometric data BD related to the pet PET. The measurement apparatus 100 may be in contact with a chest or an abdomen of the pet PET. For example, the measurement apparatus 100 may be coupled to a harness mounted on the pet PET, and may come into contact with the chest or abdomen of the pet PET. For example, the measurement apparatus 100 may be made of a material such as a string surrounding a neck, a chest, a back, a forelimb, or the like of the pet PET or a cloth enclosing an area, like a wearable method such as a wristband of a person.

The biometric data BD collected by the measurement apparatus 100 may indicate biometric information of the pet PET. According to embodiments, the biometric data BD may be data related to at least one of a heart rate, a blood pressure, a respiration rate, a pulse, temperature, acceleration, speed, and angular velocity of the pet PET.

The biometric data BD measured (or collected) by the measurement apparatus 100 may be transmitted to the server 300. According to embodiments, the measurement apparatus 100 may directly transmit the biometric data BD to the server 300, the measurement apparatus 100 may transmit the biometric data BD to the user terminal 200, or the terminal 200 may transmit the received biometric data BD to the server 300.

For example, the measurement apparatus 100 may transmit the biometric data BD to the user terminal 200 or the server 300 using short-range wireless communication or long-distance wireless communication.

The user terminal 200 is a terminal having an arithmetic processing function and a communication function, and may be a device carried or used by a user. For example, the user terminal 200 may be a smartphone, a personal computer (PC), a notebook computer, a smart watch, a tablet, or a wearable device, but embodiments of the present invention are not limited thereto.

The user terminal 200 may receive the biometric data BD transmitted from the measurement apparatus 100 and transmit the biometric data BD to the server 300.

In addition, according to embodiments, the user terminal 200 may receive the biometric data BD and generate a diagnosis result data DRES indicating the status of the pet PET using the received biometric data BD. In this case, the user terminal 200 may compare the stored average biometric data with the collected biometric data BD, and generate the diagnosis result data DRES indicating the status of the pet PET according to the comparison result. Meanwhile, according to embodiments, the user terminal 200 may receive the diagnosis result data DRES indicating the status of the pet PET from the server 300.

The user terminal 200 may visually or aurally display the diagnosis result data DRES indicating the status of the pet PET to the user.

The server 300 may receive the biometric data BD related to the pet PET, analyze the received biometric data BD, and generate the analysis result.

According to embodiments, the server 300 may generate various statistical data related to the biometric data of the pet PET by using the biometric data BD related to the pet PET. The statistical data may include reference biometric data for defining a normal range of the biometric data.

The server 300 may generate various statistical data related to the biometric data of the pet PET based on an algorithm such as a machine learning technique. For example, the server 300 may calculate an average vital sign of pet PET, but is not limited thereto.

According to embodiments, the server 300 may generate the diagnosis result data DRES indicating a health condition of the pet PET by using the biometric data BD related to the pet PET. For example, the server 300 may generate the diagnosis result data DRES indicating the health status of the pet PET based on the biometric data BD and the reference biometric data related to the pet PET. For example, the server 300 may generate the diagnosis result data DRES including health abnormality of the pet PET.

The server 300 may transmit the diagnosis result data DRES to the user terminal 200. According to embodiments, the server 300 may transmit the diagnosis result to the user terminal 200. For example, the server 300 may transmit visual data or auditory data related to the diagnosis result to the user terminal 200.

The server 300 may transmit and receive data to and from an accessible database 310. According to embodiments, the server 300 may read data stored in the database 310.

The database 310 may store reference biometric data for the pet PET. For example, the reference biometric data may be biometric data indicating a stable status of the pet PET, and may be data related to at least one of a reference vital sign and reference movement.

According to embodiments, the database 310 may match and store reference biometric data for each type, breed, and body information of the pet PET. For example, the database 310 may match and store the reference biometric data for the pet PET that is a Pomeranian (breed) dog (type) having a height of 80 cm and a weight of 2 kg (body information).

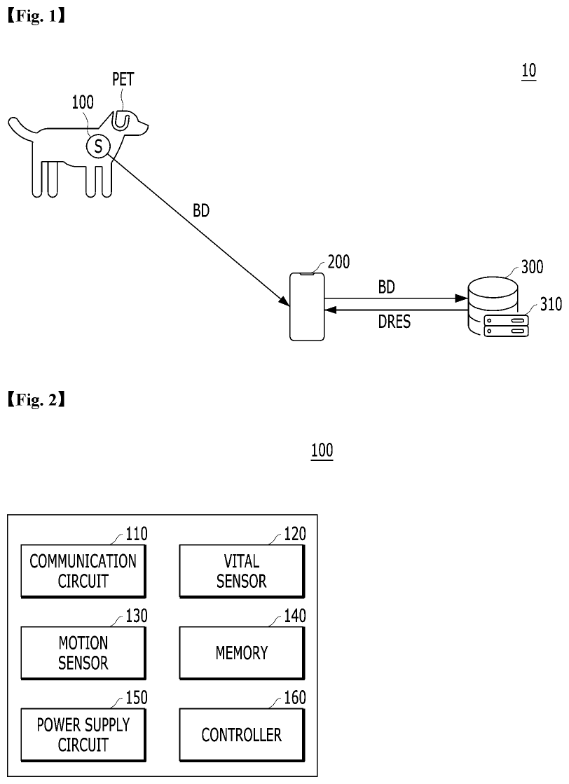

FIG. 2 is a diagram illustrating a measurement apparatus according to embodiments of the present invention. Referring to FIG. 2, the measurement apparatus 100 may include a communication circuit 110, a vital sensor 120, a motion sensor 130, a temperature sensor, a memory 140, a power supply circuit 150, and a controller 160.

The communication circuit 110 may support communication between the measurement apparatus 100 and another device. According to embodiments, the communication circuit 110 may transmit the biometric data BD generated by the measurement apparatus 100 to the user terminal 200 according to a wireless communication protocol. For example, the wireless communication protocol may include Bluetooth, radio frequency identification (RFID), infrared data association (IrDA), ultra-wideband (UWB), ZigBee, near field communication (NFC), ultrasonic sound communication (USC), visible light communication (VLC), Wi-Fi, Wi-Fi direct, etc., but the embodiments of the present invention are not limited thereto.

Also, according to embodiments, the measurement apparatus 100 may transmit the biometric data BD to the server 300 according to the wireless communication protocol.

The vital sensor 120 may be configured to measure a vital sign of the pet PET. The vital sign may include, but are not limited to, at least one of a heart rate, a blood pressure, a respiratory rate, a pulse, and body temperature. The vital sensor 120 may generate vital data indicating the vital sign of the pet PET according to the measurement result. For example, the vital data may include at least one of heart rate data, blood pressure data, respiration rate data, pulse data, and body temperature data.

According to some embodiments, the vital sensor 120 may measure a heart sound of the pet PET and calculate the heart rate of the pet PET based on the measured heart sound. For example, the vital sensor 120 may include an acoustic type heart rate sensor or a UWB type heart rate sensor, but is not limited thereto.

For example, the vital sensor 120 may include a microphone (e.g., a piezo microphone) configured to measure sound and a processor configured to process the measured sound, and the processor may calculate the heart rate from the heart sound by filtering, as noise, remaining sounds other than a sound of a specific frequency band or a specific beat tempo (beats per minute) band from among the measured heart sounds.

According to embodiments, the vital sensor 120 may measure an acceleration of a diaphragm or an abdomen of the pet PET and analyze the measured acceleration of the diaphragm or abdomen, thereby generating respiration rate data indicating the respiration rate of the pet PET. For example, the vital sensor 120 may include a respiration rate sensor of a UWB method or a 6-axis acceleration sensor method, but embodiments of the present invention are not limited thereto.

According to embodiments, the vital sensor 120 may measure the body temperature of the pet PET, and may generate temperature data according to the measurement result. For example, the vital sensor 120 may include an infrared temperature sensor, but embodiments of the present invention are not limited thereto.

The motion sensor 130 may measure at least one of acceleration, speed, angular velocity, and movement distance of the pet PET. Also, the motion sensor 130 may generate acceleration data, velocity data, angular velocity data, and movement distance data indicating each of the measured acceleration, velocity, angular velocity, and movement distance.

Also, the motion sensor 130 may calculate an extent (e.g., activity amount) of movement of the pet PET based on at least one of the measured acceleration, speed, angular velocity, and movement distance, and generate the movement data indicating the movement.

For example, the motion sensor 130 may be configured as a sensor module including at least one of a speed sensor, an acceleration sensor, and a gyro sensor, but embodiments of the present invention are not limited thereto.

That is, the biometric data BD generated by the measurement apparatus 100 according to embodiments of the present invention may include at least one of vital data and movement data.

The memory 140 may store data necessary for the operation of the measurement apparatus 100. According to embodiments, the memory 140 may store the biometric data BD. For example, the memory 140 may be implemented as a volatile memory or a non-volatile memory, but is not limited thereto.

Although only one memory 140 is illustrated in FIG. 2, according to embodiments, the measurement apparatus 100 may include a plurality of memories, and each of the plurality of memories may be integrally with the sensors 120, 130 and 140 and the controller 160, but embodiments of the present invention are not limited thereto.

The power supply circuit 150 may supply power necessary for the operation of the measurement apparatus 100. According to embodiments, the power supply circuit 150 may supply DC power to each of the components 110, 120, 130, 140, 150 and 170 of the measurement apparatus 100.

For example, the power supply circuit 150 may include an AC-DC converter for converting AC power supplied from the outside into DC power. In addition, the power supply circuit 150 may include a battery configured to store DC power.

The controller 160 may control the overall operation of the measurement apparatus 100. According to embodiments, the controller 160 may read the biometric data BD generated by the sensors 120, 130 and 140 from the memory 140, and transmit the biometric data BD to the outside through the communication circuit 110.

Also, the controller 160 may control the power supply circuit 150. According to embodiments, the controller 160 may adjust DC power supplied from the power supply circuit 150 to each of the components 110, 120, 130, 140, 150, and 170 of the measurement apparatus 100, or control whether to supply the DC power. That is, the power supply circuit 150 may supply or may not supply the DC power to each of the components 110, 120, 130, 140, 150, and 170 of the measurement apparatus 100 according to the control of the controller 160.

Although the communication circuit 110 and the controller 160 are illustrated separately in FIG. 2, the communication circuit 110 may be implemented integrally with the controller 160 according to embodiments.

Meanwhile, in order to measure the vital sign of the pet PET, it is desirable to measure the vital sign during normal sleep or rest time because the stable status of the pet PET is required. On the other hand, when the movement of the pet PET is large, it is preferable not to measure the vital sign. In this case, the measured vital sign does not accurately indicate a health condition of the pet PET. That is, when the movement of the pet PET is large, the measured vital sign may have low utilization.

Accordingly, according to embodiments of the present invention, it may vary whether or not the measurement apparatus 100 performs the operation of the measurement operation of the vital sign of the pet PET according to the movement of the pet PET. Accordingly, only vital data well indicating the health condition of the pet PET may be acquired.

In addition, since the vital sensor 120 may be inactive when the movement of the pet PET is large, the power consumption by the operation of the vital sensor 120 may be reduced, and thus, the operation time of the measurement apparatus 100 may be further increased. In particular, since the measurement apparatus 100 is a wearable apparatus that is directly worn on the pet PET, as the operation time of the measurement apparatus 100 further increases, the number of times of charging is reduced and portability can be further maximized.

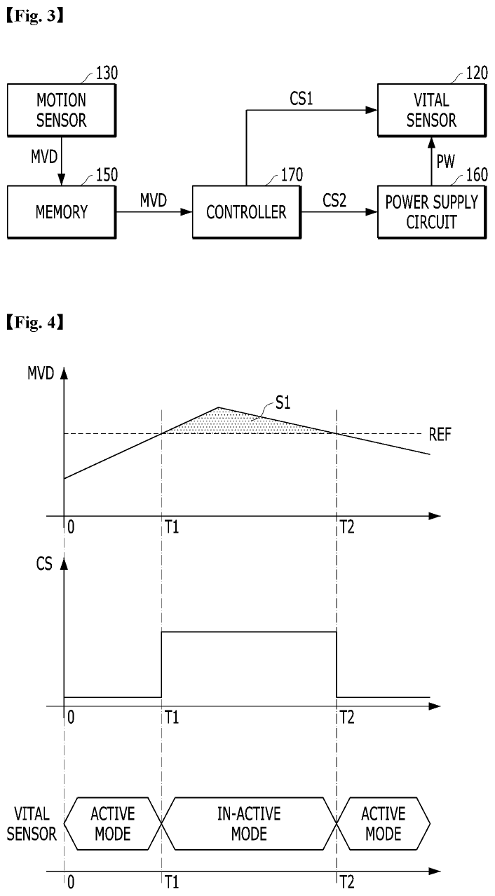

FIG. 3 illustrates a part of the measurement apparatus according to embodiments of the present invention. Referring to FIG. 3, the controller 160 may control the operation of the vital sensor 120 based on the movement data generated by the motion sensor 130.

The motion sensor 130 may measure the movement of the pet PET and generate movement data (MVD) indicating the movement of the pet PET. According to embodiments, the motion sensor 130 may measure at least one of the speed, acceleration, angular velocity, and movement distance of the pet PET, and may generate the movement data (MVD) indicating at least one of the measured values. The generated movement data MVD may be stored in the memory 140.

The controller 160 may perform operations for controlling the operation of the vital sensor 120 based on the movement data MVD. For example, the controller 160 may perform an operation for reducing power consumed by the vital sensor 120 based on the movement data MVD.

According to embodiments, the controller 160 may output, to the vital sensor 120, a first control signal CS1 for setting the operation mode of the vital sensor 120 to any one of an active mode and an inactive mode based on the movement data (MVD). The vital sensor 120 may operate according to any one of the active mode and the inactive mode in response to the first control signal CS1.

The active mode may be a mode in which the vital sign measurement operation of the vital sensor 120 is performed (i.e., the vital data is generated), and the inactive mode is a mode in which the vital sign measurement operation of the vital sensor 120 may not be performed (i.e., the vital data is not generated). In this case, the power consumption of the vital sensor 120 in the inactive mode may be smaller than that of the vital sensor 120 in the active mode.

According to embodiments, the controller 160 may output a second control signal CS2 for controlling power PW supplied from the power supply circuit 150 to the vital sensor 120 based on the movement data MVD to the power supply circuit 150. The power supply circuit 150 may or may not supply the power PW to the vital sensor 120 in response to the second control signal CS2.

For example, the power supply circuit 150 may include a switch circuit that is connected to the vital sensor 120 and selectively supplies power to the vital sensor 120, in which the switch circuit may be turned on and turned off in response to the second control signal CS.

FIG. 4 is a diagram for describing the operation of the controller according to embodiments of the present invention. Referring to FIG. 4, the movement data MVD, the level of the control signal CS, and the status of the vital sensor are shown.

According to embodiments, the controller 160 may compare the movement data MVD with a reference value REF, and generate control signals CS1 and CS2 (collectively, CS) for controlling the vital sensor 120 according to the comparison result. The reference value REF may be a value indicating the movement of the pet in a status suitable for measuring the vital sign of the pet.

In first section 0 to T1, the movement data MVD generated by the motion sensor 130 may be equal to or less than the reference value REF. In this case, the controller 160 may output a control signal CS of a first level (e.g., low level) according to the result of comparing the movement data MVD and the reference value REF. The reference value REF may indicate the movement of the pet PET in the status suitable for measuring the vital sign.

For example, the vital sensor 120 may operate in the active mode in response to the control signal CS of the first level. Also, for example, the power supply circuit 150 may supply the power PW to the vital sensor 120 in response to the control signal CS of the first level.

That is, when the movement data MVD generated by the motion sensor 130 is equal to or less than the reference value REF, the vital sensor 120 operates normally, and the vital sensor 120 generates the vital data indicating the vital sign of the pet PET.

In second section T1 to T2, the movement data MVD generated by the motion sensor 130 may exceed the reference value REF. In this case, the controller 160 may output a control signal CS of a second level (e.g., high level) according to the result of comparing the movement data MVD and the reference value REF.

For example, the vital sensor 120 may be switched from an active mode to an inactive mode in response to the control signal CS of the second level, and may operate in an inactive mode. Also, for example, the power supply circuit 150 may cut off the power PW supplied to the vital sensor 120 in response to the control signal CS of the second level.

That is, when the movement data MVD generated by the motion sensor 130 exceeds the reference value REF, the vital sensor 120 may operate or may not operate in the inactive mode, and thus, may generate the vital data indicating the vital sign of the pet PET.

In a third section (after T2), the movement data MVD generated by the motion sensor 130 may be equal to or less than the reference value REF. In this case, the controller 160 may output the control signal CS of the first level according to the result of comparing the movement data MVD and the reference value REF.

Similarly, when the movement data MVD generated by the motion sensor 130 is equal to or less than the reference value REF, the vital sensor 120 operates normally, and the vital sensor 120 generates the vital data indicating the vital sign of the pet PET.

As described with reference to FIG. 4, when the movement data MVD generated by the motion sensor 130 exceeds the reference value REF, the vital sensor 120 may not generate heartbeat data under the control of the controller 160, and as a result, the power consumed by the vital sensor 120 may be reduced. Therefore, according to embodiments of the present invention, when the movement of the pet PET is large, the measurement apparatus 100 may not generate inaccurate vital data, and the usage time of the measurement apparatus 100 may be maximized by preventing unnecessary power consumption.

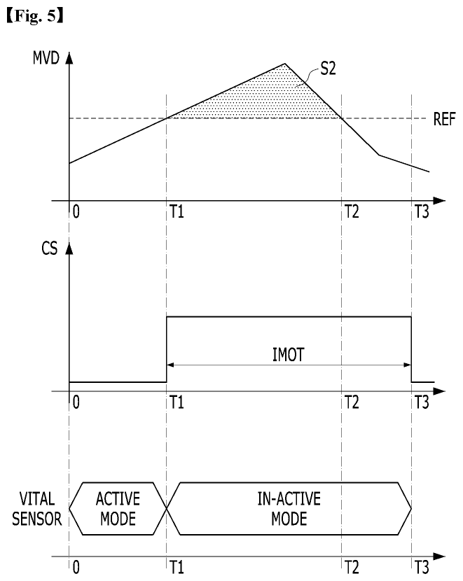

FIG. 5 is a diagram for describing the operation of the controller according to embodiments of the present invention. Referring to FIG. 5, the movement data MVD, the level of the control signal CS, and the status of the vital sensor are shown.

According to embodiments, the controller 160 may compare the movement data MVD with the reference value REF, and generate the control signal for controlling the vital sensor 120 according to the comparison result.

For example, as illustrated in FIG. 5, in the first section 0 to T1, the movement data MVD generated by the motion sensor 130 may be equal to or less than the reference value REF. In this case, the controller 160 may output a control signal CS of a first level (e.g., low level) according to the result of comparing the movement data MVD and the reference value REF.

For example, the vital sensor 120 may operate in the active mode in response to the control signal CS of the first level. Also, for example, the power supply circuit 150 may supply the power PW to the vital sensor 120 in response to the control signal CS of the first level.

Meanwhile, in the second section T1 to T2, the movement data MVD generated by the motion sensor 130 may exceed the reference value REF. In this case, the controller 160 may output a control signal CS of a second level (e.g., high level) according to the result of comparing the movement data MVD and the reference value REF.

For example, the vital sensor 120 may be switched from an active mode to an inactive mode in response to the control signal CS of the second level, and may operate in an inactive mode. Also, for example, the power supply circuit 150 may cut off the power PW supplied to the vital sensor 120 in response to the control signal CS of the second level.

In a third section (after T2), the movement data MVD generated by the motion sensor 130 may be equal to or less than the reference value REF. However, unlike FIG. 4, even if the movement data MVD is equal to or less than the reference value REF, the controller 160 may not output the control signal CS of the first level.

In the embodiment of FIG. 5, the controller 160 may determine an inactive mode operation time (IMOT) of the vital sensor 120 based on the extent to which the movement data MVD exceeds the reference value REF and the length of the section in which the movement data MVD exceeds the reference value REF. When the inactive mode operation time (IMOT) is exceeded from the time when the control signal CS of the second level is output, the controller 160 may output the control signal CS of the first level.

For example, as illustrated in FIG. 5, the controller 160 may determine the inactive mode operation time (IMOT) based on an area S2 of the section in which the movement data MVD exceeds the reference value REF. That is, according to embodiments of the present invention, as the movement data MVD exceeds the reference value REF more and longer, the time during which the vital sensor 120 operates in the inactive mode becomes longer. The fact that the movement data (MVD) exceeded the reference value REF more and longer means that the movement of the pet PET is very active, so it will take longer for the pet PET to rest for a longer period to return to its stable status. Therefore, the vital data measured by the vital sensor 120 after a longer time may more accurately indicate the vital sign of the pet PET.

For example, the area S2 of the section in which the movement data MVD in FIG. 5 exceeds the reference value REF is smaller than the area S2 of the section in which the movement data MVD in FIG. 4 exceeds the reference value REF, the inactive mode operation time (IMOT) in FIG. 5 may be longer than in FIG. 4.

According to embodiments, the controller 160 may directly calculate the inactive mode operation time (IMOT) from the extent to which the movement data MVD exceeds the reference value REF and the length of the section in which the movement data MVD exceeds the reference value REF, or may acquire the inactive mode operation time (IMOT) from a pre-stored lookup table.

Therefore, according to embodiments of the present invention, when the movement of the pet PET is large, the measurement apparatus 100 may not generate inaccurate vital data, and the usage time of the measurement apparatus 100 may be maximized by preventing unnecessary power consumption.

In addition, according to embodiments of the present invention, when the movement of the pet PET exceeds a predetermined reference, the inactive operation time of the vital sensor 120 based on the extent to which the movement exceeds the reference and the length of time when the movement exceeds the reference may be determined, so it is possible to acquire more accurate vital data.

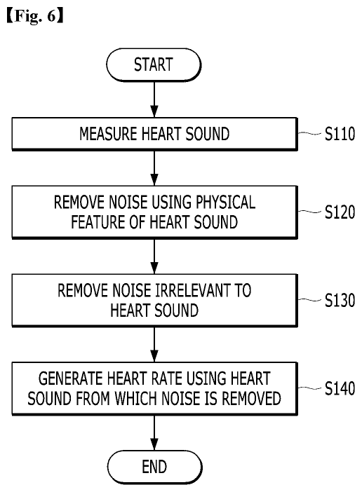

FIG. 6 is a flowchart illustrating a method of measuring a heart rate according to embodiments of the present invention. The method of measuring a heart rate described with reference to FIG. 6 may be performed by the measurement apparatus 100 described with reference to FIG. 1. In addition, the method of measuring a heart rate may be implemented in the form of a program stored in a computer-readable non-transitory storage medium.

Referring to FIG. 6, the measurement apparatus 100 may measure the heart sound of the pet PET and acquire a heart sound signal related to the heart sound (S110).

In order to accurately measure the heart sound of the pet PET, it is preferable that friction between the measurement apparatus 100 and the pet PET is excluded altogether. However, due to the characteristics of the measurement apparatus 100, which is a wearable device, a sound caused by rubbing the skin, hair, etc., of the pet PET on the material surrounding the surface of the measurement apparatus 100 or the vital sensor 120 may be generated as the largest noise. Therefore, in order to listen to a specific sound (heartbeat, etc.) inside the body of the pet PET as intended, it is necessary to derive the unique properties of the heartbeat sound, which is distinct from other sounds such as noise, and use the derived properties to remove the noise.

The measurement apparatus 100 may remove noise by using the physical characteristics of the heart sound (S120). According to embodiments, the measurement apparatus 100 may remove noise by using physical characteristics (e.g., frequency and pulse characteristics) of the heart sound. For example, the measurement apparatus 100 may filter, as noise, a sound other than a sound of a specific frequency band or a specific beat tempo band from among the measured heart sounds.

For example, the measurement apparatus 100 may extract a sound in a range of 10 Hz to 250 Hz and 80 bpm to 100 bpm of the heart sound by removing noise of the measured heart sound.

The measurement apparatus 100 may remove noise irrelevant to the heart sound from the heart sound (S130). For example, the measurement apparatus 100 may remove noise by complexly using various algorithms for analyzing audio. For example, the measurement apparatus 100 may remove noise by using a drum transcription technique, an audio separation technique, and algorithms such as RNN, LSTM, and DBN.

The measurement apparatus 100 may generate heart rate data using heart sound from which noise is removed (according to S120 and S130) (S140).

According to embodiments of the present invention, since the heart sound from which the noise is removed using the physical characteristics of the measured heart sound and the heart rate of the pet PET may be measured using the heart rate of the pet PET, it is possible to generate the heart rate data more accurately indicating the heart rate of the pet PET.

Although the present specification has been described with reference to exemplary embodiments shown in the accompanying drawings, it is only an example. It will be understood by those skilled in the art that various modifications and other equivalent exemplary embodiments are possible from the present invention. Accordingly, an actual technical protection scope of the present specification is to be defined by the technical spirit of the following claims.

### INDUSTRIAL APPLICABILITY

Embodiments of the present invention relate to an apparatus for measuring biometric information of a pet.

1. A measurement apparatus for measuring biometric information of a pet, comprising:
a vital sensor configured to measure a vital sign of the pet and generate vital data indicating the vital sign;
a motion sensor configured to measure movement of the pet and generate movement data indicating the movement;
a memory configured to store the vital data and the movement data;
a communication circuit configured to transmit the vital data and the movement data to an external apparatus; and
a controller configured to control the measurement apparatus,
wherein the controller controls an operation mode of the vital sensor to be any one of an active mode and an inactive mode, on the basis of the movement data measured by the motion sensor, and
the vital sensor does not generate the vital data in the inactive mode.

2. The measurement apparatus of claim 1, wherein the vital sensor includes:
a microphone configured to be in contact with the pet and measure a heart sound of the pet; and
a processor configured to generate heart rate data indicating the heart rate of the pet by processing the heart sound measured by the microphone.

3. The measurement apparatus of claim 2, wherein the processor generates the heart rate data by filtering, as noise, remaining sounds other than for a sound of a specific frequency band or a specific beat tempo band from among the measured heart sounds.

4. The measurement apparatus of claim 1, wherein the motion sensor measures at least one of speed, acceleration, and angular velocity of the pet, and generates movement data indicating the at least one.

5. The measurement apparatus of claim 1, wherein power consumption of the vital sensor in the active mode is greater than that of the vital sensor in the inactive mode.

6. The measurement apparatus of claim 1, wherein the controller compares the movement data with a reference value indicating the movement of the pet in a status suitable for measuring a vital sign, and
when the movement data exceeds the reference value, the operation mode of the vital sensor is controlled to be in the inactive mode.

7. The measurement apparatus of claim 6, wherein, when the movement data exceeds the reference value, the controller outputs, to the vital sensor, a first control signal for controlling the operation mode of the vital sensor to be in the inactive mode, and
the vital sensor operates in the inactive mode in response to the first control signal.

8. The measurement apparatus of claim 6, further comprising:
a power supply circuit configured to supply power to the vital sensor,
wherein, when the movement data exceeds the reference value, the controller outputs, to the power supply circuit, a second control signal for cutting off the supply of power from the power supply circuit to the vital sensor, and
the power supply circuit cuts off the supply of power to the vital sensor in response to the second control signal.

9. The measurement apparatus of claim 6, wherein the controller determines an inactive mode operation time when the vital sensor operates in the inactive mode based on at least one of an extent in which the movement data exceeds the reference value and a length of a section in which the vital sensor exceeds the reference value, and
controls the operation mode of the vital sensor to be in the active mode after the inactive mode operation time has elapsed.
